(19) United States
(12) Patent Application Publication
(10) Pub. No.: US 2003/0044789 A1
(43) Pub. Date: Mar. 6, 2003

(54) Identification and quantification of a protein carrying an N-terminal polyhistidine affinity tag

(76) Inventors: Thomas J. Burke, Madison, WI (US); Robert Lowery, Brooklyn, WI (US); Karen Kleman-Leyer, Madison, WI (US)

Correspondence Address: Andrew S. Marks, Vertex Pharmaceuticals Inc., 130 Waverly Steet, Cambridge, MA, 02139-4242, US

(21) Appl. No.: 09/875,620

(22) Filed: Jun. 6, 2001

Related U.S. Application Data
(60) Provisional application No. 60/209,684, filed on Jun. 6, 2000.

(51) Int. Cl. C12Q 1/68

(52) U.S. Cl. 435/6

(57) ABSTRACT

A rapid fluorescence polarization immunoassay (FPIA) for accurate quantification of any protein carrying an N-terminal polypeptide affinity tag especially a polyhistidine affinity tag (HisTag).

### CROSS-REFERENCE TO RELATED APPLICATIONS

This application is related to provisional application Serial No. 60/209,487 filed on Jun. 6, 2000.

### FEDERALLY SPONSORED RESEARCH

N/A

### FIELD

This invention is related to detecting and quantitating proteins in a solution utilizing a novel approach.

### BACKGROUND

To realize the biomedical potential of human genome sequence and expression data—an estimated 3,000-10,000 new drug targets over the next ten years—high throughput approaches are needed for isolating individual proteins in an active form and elucidating their functions. Expression of recombinant human proteins in a soluble, active form is still an empirical process, thus rapid methods for assessing multiple expression parameters for each protein are needed. This application addresses one of the major bottlenecks preventing increased throughput at the protein expression level: quantitative detection of diverse proteins in crude cell extracts. Existing methods such as gel electrophoresis, immunoblotting and mass spectrometry are too labor intensive for a high throughput format and are not highly quantitative.

It is not practical to use a protein-specific approach such as direct immunodetection or activity assays with large numbers of diverse proteins because the required reagents and assay conditions vary for different protein families, and often even for individual members of a class. In fact antibodies, specific assays, and reference activity levels will not be available for the many uncharacterized proteins that are coming out of genomics efforts. SDS-PAGE and mass spectral analysis do not require reagents specific to a particular protein or class of proteins, and are both used for detection and, to some degree quantification, of any protein present in a crude mixture. Unfortunately, these methods require relatively cumbersome sample preparations and manipulations that are difficult to incorporate into a high throughput approach. In addition, quantification using these approaches suffers from protein-specific differences in the assay signals: staining intensity of electrophoresed proteins and the ion spectra for proteins both vary depending on structure.

### SUMMARY

To overcome these technical barriers, we have developed a rapid fluorescence polarization immunoassay (FPIA) for accurate quantification of any protein carrying an N-terminal polyhistidine affinity tag (HisTag).

Fusion of a HisTag comprised of 4-10 histidine residues to a recombinant protein, most commonly to the N-terminus, provides a common “handle” for diverse proteins that generally allows substantial purification in a single chromatographic step and rarely interferes with protein function. For this reason, HisTags are used extensively—more than any other affinity tag—by both academic and industry researchers to simplify production of proteins for functional characterization and for use in HTS drug screening assays. We would suggest that it could be used successfully for expression of most novel cDNAs. Binding of the histidines to immobilized metal ion adsorbents, known as immobilized metal ion chromatography (IMAC), provides a powerful affinity purification method that can be used even in the presence high salt, detergent or denaturants followed by selective elution using imidazole. We have expressed a broad range of proteins as HisTag fusions including nuclear receptors, protein kinases, protein tyrosine phosphatases, proteases, cytochromes P450, and growth factors without a deleterious effect on function. As described in more detail below, the small size of the HisTag also makes it ideal for fluorescent labeling and use as an FPIA tracer because the assay signal is a function of the difference in size between the tracer and the Ab.

The idea of using an affinity tag to simplify detection of diverse proteins is not novel, however the approaches used previously were not suitable for rapid and accurate assessment of multiple expression parameters in a high throughput format. For instance HisTags and other N-terminal fusions can be quantified by immunodetection methods such as Western blotting or ELISA; these methods have been available for several years. However both of these methods require multiple binding and wash steps and are difficult to calibrate—Western blotting also requires prior gel electrophoresis and electroblotting of samples. The HisTag FPIA overcomes these detection problems, and though there are some significant advantages to the use of HisTags over other affinity tags, the FPIA approach could be applied to any protein domain used as a fusion including glutathione transferase (GST), maltose binding protein (MBP) and thioredoxin (TRX), various immunoaffinity domains (Myc, FLAG), carbohydrate binding domains (MBP, Chitin-binding), and protein binding domains (RNase S-peptide).

### BRIEF DESCRIPTION OF THE DRAWINGS

FIG. 1 is a competitive FPIA for HisTagged proteins. Prior to the addition of sample, most of the fluorescent HisTag tracer peptide is bound to Ab, causing a high polarization because of the large effective molecular volume of the complex. Addition of sample, a HisTagged protein, results in displacement of the tracer peptide from the Ab, thus decreasing the polarization. Competitive binding data, including the fraction of the probe bound at various competitor concentrations, are generated using FP and analyzed graphically in a manner similar to that used for radioactive ligand binding assays.

FIG. 2 shows binding of TetraHis Ab and PentaHis Ab to fluorescein-labeled HisTag peptides.

FIG. 3 illustrates competitive binding of unlabeled HisTag Peptides. Unlabeled peptide (AHHHHHHG) was serially diluted in PBS +0.01% NP-40 from a concentration of 400 uM to 4.76×10⁻⁵ uM. Fluorescein labeled peptide (1 nM, fluor-AHHHHHHG) and 10 nM of antibody (Tetra-His or Penta-His) were then added to each reaction in multiwell plates. After a five minute incubation at room temperature, the plates were read on a Tecan Polarian instrument. The IC₅₀ values were calculated from the competition curves using nonlinear least-squares curve fitting using Prism (Graphpad; SanDiego, Calif.).

FIG. 4 shows a competition Study with HisTagged Proteins. Purified N-terminal HisTagged proteins were serially diluted into PBS +0.01% NP-40 in a multiwell plates. 1-2 nM fluorescein labeled peptide (fluor-AHHHHHHG) and 10 nM of Penta-His antibody were then added to each reaction to a final volume of 50-100 ul. After approximately 60 min incubation at room temperature to insure equilibrium binding, the polarization was measured on a Tecan Polarian multiwell instrument. The IC₅₀ values were calculated from the competition curve using nonlinear least-squares curve fitting using Prism (Graphpad; SanDiego, Calif.).

FIG. 5 shows a strategy for testing different linker peptides for preventing context specific Ab binding to HisTagged fusion proteins. Expressed HisTag-Linker-Reporter constructs are assayed in crude extracts using the HisTag FPIA and quantified independently using FP-based tyrosine kinase activity assays developed at PanVera. The assay response (ΔmP/pmol) for each HisTag-Linker-Reporter construct are compared with the response for a HisTag peptide calibration standard. This allows identification of those linker peptides which allow the Ab to bind the HisTag domain with equal affinity regardless of the structure of the protein to which it is fused.

### DETAILED DESCRIPTION

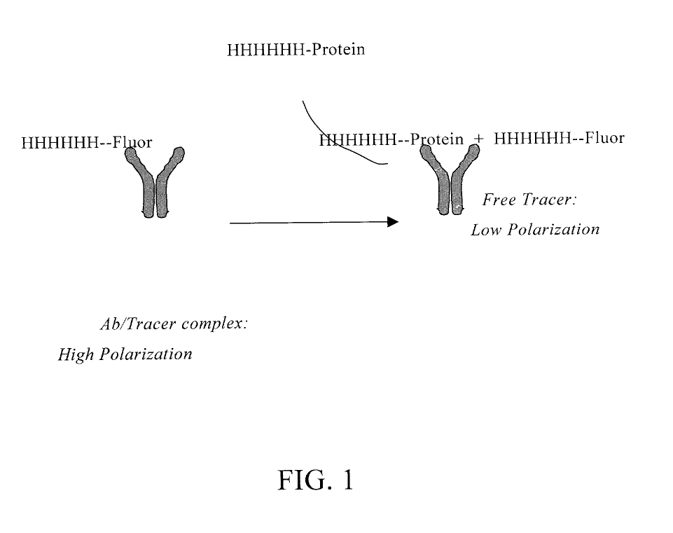

The HisTag FPIA provides the ability to quantify the expression level of diverse expressed proteins in crude cell extracts using a simple “mix and read” format; i.e., by adding samples to a sample tube or multiwell plates and obtaining a fluorescence polarization reading. The assay operates by the same basic principles as a competitive binding assay: the sample molecules, His-Tagged proteins, compete with a tracer, in this case a fluorescently labeled HisTag peptide, for binding to Ab (FIG. 1). Displacement of the fluorescent tracer from the Ab results in a decrease in its fluorescence polarization value, which can be measured in a multiwell plate reader. The simplicity of the assay principle and methods make it especially well suited for an automated HTS format.

The HisTag FPIA allows:

high throughput expression screening of crude cell extracts

from different host organisms

expressing diverse HisTagged proteins

using a homogenous (single addition) assay method with one calibration standard.

To fully explain how fluorescence polarization can be used to overcome the shortcomings of existing protein detection methods, it is helpful to briefly summarize the principles of the technique. Fluorescence polarization (FP) is used to study molecular interactions by monitoring changes in the apparent size of fluorescently-labeled or inherently fluorescent molecules. When a small fluorescent molecule (ligand) is excited with plane polarized light, the emitted light is largely depolarized because the molecule rotates rapidly in solution within the timescale of the fluorescence event, or the time between excitation and emission. However, if the fluorescent ligand is bound to a much larger receptor, thereby increasing its effective molecular volume, its rotation is slowed sufficiently to emit light in the same plane in which it was excited. The bound and free states of the fluorescent molecule each have an intrinsic polarization value, a high value for the bound state and a low value for the free state. In a population of molecules, the measured polarization is a weighted sum of the two values, thus providing a direct measure of the fraction of the fluorescent molecule which is bound. Competitive binding data, including the fraction of the probe bound at various competitor concentrations, are generated using FP and analyzed graphically in a manner similar to that used for radioactive ligand binding assays.

FP offers several key advantages over other technologies for detection of affinity tagged proteins in an HTS format. Specifically, FP is:

Homogenous--FP is a homogenous or “addition only” assay format: it does not require separation of free from bound ligand, thus can be performed by a single addition of sample to multiwell plates. The fluorescent probe molecule and the test compound are added to the antibody to form a mixture which is allowed to reach equilibrium and then measured with no further manipulations. This eliminates the need to attach the antibody to a solid phase and any centrifugation, blotting, filtration, and wash steps, making the technology considerably less cumbersome than existing methods, and thus much easier to format for HTS.

Accurate-Accurate quantification by any immunodetection procedure is dependent upon allowing an Ab-antigen mixture to reach an equilibrium binding state. Most immunodetection methods such as ELISA or Western blotting require immobilization and wash steps, which can result in the loss of bound antigen. Because FP allows measurement of true equilibrium binding in solution, the estimates of antigen are more accurate than with these other methods.

High Throughput-FP has become one of the major HTS platforms used by pharmaceutical companies. Three different companies, LJL, BMG and Tecan now manufacture multiwell FP instruments capable of using up to 1536 well plates, and the homogenous nature of the assay method makes it well suited for coupling to automated dispensing stations.

High Sensitivity-The sensitivity of a competitive FPIA is a function of antibody affinity and fluor intensity. The generation of antibodies with subnanomolar affinity for tracer and the use fluorescein, one of the brightest fluors known, for synthesis of tracers, makes if feasible to develop of FPIAs capable of detecting nanomolar quantities of sample. This is sufficient for detection of very low levels (i.e., less than 1 mg/liter) of expressed proteins. In addition, because FP is a ratio of fluorescence intensities in two planes, decreases in intensity due to light scattering or absorption are not as serious an issue as with direct intensity measurements. This makes FP well suited for measurements in crude extracts.

Universal and quantitative: A desirable feature of an HisTag-FPIA is that it provides a single rapid method for quantifying the expression level of any protein, regardless of its structure or function—a universal assay, which can be calibrated using a single HisTag-peptide standard; i.e., the affinity of the Ab for the HisTag can be the same whether it is a free peptide or an N-terminal affinity tag. This is possible because any context specificity in the binding of the antibody to the HisTags on the expressed proteins can be eliminated by spatially separating the HisTag from the protein to which it is attached.

### EXAMPLES

### Example 1

Binding of Antibodies to Fluorescein-Labeled HisTag Peptides

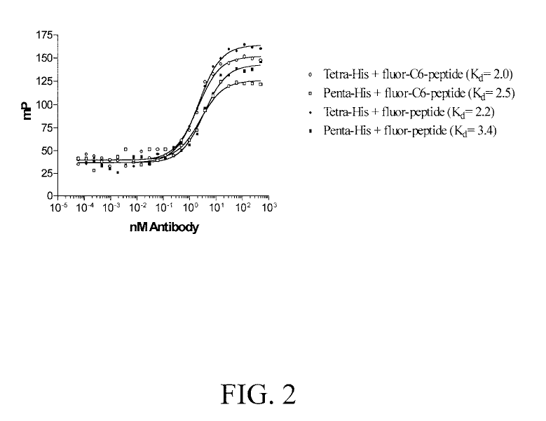

The premise of the proposed competitive FPIA for detecting HisTagged proteins is the displacement of a fluorescent peptide (tracer) from a tightly bound specific antibody (FIG. 1). The first step in demonstrating the feasibility for this, or any FPIA, is to show an increase in polarization when Ab binds to tracer. This was demonstrated by synthesizing an “AHHHHHHG” peptide with fluorescein at the N-terminus with and without a six carbon linker, incubating each of them with different amounts of Penta-His or Tetra-His antibody (Qiagen), and reading the corresponding fluorescence polarization on a Tecan Polarian instrument. As shown in FIG. 2, binding of either Ab caused an increase of 80-120 mP in the polarization value for the tracer. Dissociation constants were calculated from the binding isotherms shown in FIG. 2 using Prizm software from Graphpad; Tetra-His and Penta-His antibodies bind to the various fluorescein-labeled peptides tested with the K_{d} vaules ranging from 2.0-3.4 nM. The optimum antibody/tracer combination was Penta-His antibody with the peptide labeled directly at the N-terminus (K_{d}=2.0, delta mP=120).

500 nM of each antibody was serially diluted in PBS +0.01% NP-40 to the concentrations indicated in FIG. 2. Fluorescein-labeled peptides (AHHHHHHG) with (fluor-C6-peptide) or without a six carbon linker (fluor-peptide) were added to a final concentration of 1 nM in a final volume of 200 ul in 96-well plates. After room temperature incubation, polarization was measured using a multiwell Tecan Polarian instrument (excitation 485 nm, emission 530 nm). To calculate K_{d} values, the competition curves were analyzed by nonlinear least-squares curve fitting using Prism software (Graphpad; SanDiego, Calif.)

### Example 2

Competition Studies with Unlabeled HisTag peptide.

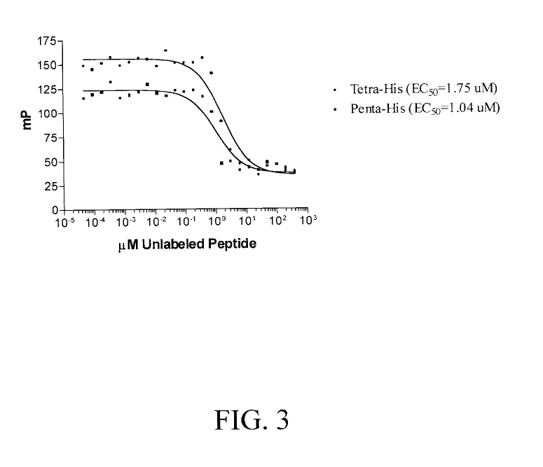

After demonstrating an FP increase with Ab/tracer binding, the next feasibility issue for an FPIA is competition with unlabeled antigen; i.e., the ability of sample molecules to displace the tracer from the Ab resulting in a decrease in polarization. This was first demonstrated using purified unlabeled HisTag peptides. As shown in FIG. 3, the polarization value decreases from ˜150 to 40 mP with addition of the Tetra-His antibody and from ˜125 to 40 mP with the Penta-His antibody. These data demonstrate the specificity of the antibodies to the HisTag peptide and establishes the feasibility of detecting and quantifying HisTags in a homogenous high throughput assay format. The K_{d} values, calculated using the Graphpad Prism computer program, were 437 nM and 261 nM, respectively. Note that these K_{d} values indicate affinity between the Ab and unlabeled HisTags that is about 100-fold lower than that for the fluorescein labeled peptides. This anomaly most likely is due to a non-antigenic interaction of the fluorescein with the Ab; both PanVera and other investigators have observed this phenomenon previously.

### Example 3

Detection of HisTagged Proteins

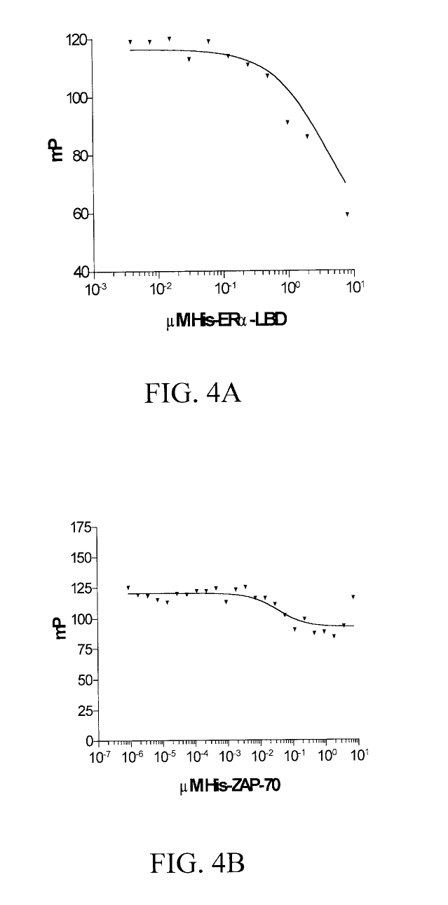

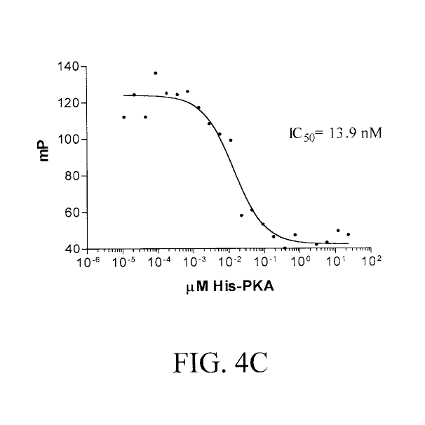

We were able to demonstrate competitive binding of three recombinant human proteins with N-terminal fusions comprised of six histidine residues (FIG. 4, below), which is the most commonly used HisTag. Protein Kinase A (PKA) is a serine/threonine kinase, ZAP-70 is a tyrosine kinase, and the Estrogen Receptor Ligand Binding Domain (ER-LBD) is comprised of the C-terminal 300 amino acids of ERα; all three proteins were highly purified and active. These experiments clearly demonstrate the potential for using the HisTag FPIA for detection of diverse proteins. The approximate IC₅₀ values (concentration of compeititor required to decrease FP signal by 50%) were 4.2 μM, 36 nM, and 14 nM for ER-LBD, ZAP-70, and PKA, respectively, and the total change in polarization for the tracer ranged from 25 mP for the ER-LBD to 80 mP for PKA.

Structurally diverse HisTag peptides, all of which have desirable expression and/or purification properties when used as N-terminal fusions, can be used in an FPIA (see Table 1 for representative examples). Whereas HisTags containing exclusively histidine residues, numbering from four to ten, are widely used in commercially available expression vectors, these are not necessarily the optimal affinity tags for IMAC; the penultimate peptide shown in Table 1, which contains only two histidines, was selected using phage display for high affinity to an immobilized Cu²⁺ column , and allowed more selective elution from IMAC that a 6xHis peptide. In addition, the inclusion of amino acids other than histidine increases the antigenic diversity of the HisTag peptides. This is desirable not only because it increases the probability of generating high affinity antiserum, but also because the increased the number of haptens increases the potential number individual Ab clones in the polyclonal serum thus resulting in higher avidity binding to the antigen. A HisTag flanked on both sides by other amino acids (peptide #2) provides a constant context for Ab binding and thereby prevents non-specific interactions of Ab with downstream protein sequences. The “GluAsp” peptide shown in Table 1 contains no histidines; its inclusion is based on studies showing that surface accessible Glu-Asp clusters can also bind with high affinity to certain IMAC adsorbents.

TABLE 1
HisTag peptides and variants that can be used in
an FPIA.

| HisTags | Desirable Properties |
|---|---|
| 1. AHHHHHHG- | Similar to existing |
| | HisTag antigens |
| 2. GSGSHHHHHHGSGS- | Imbedded HisTag to |
| | create constant con- |
| | text for Ab binding |
| 3. RHHHHHH- | Arg codon on N-ter- |
| | minus resulted in in- |
| | creased expression |
| | level |
| 4. KHQHQHQHQHQHQ- | Inclusion of alter- |
| | nating glutamines or |
| | alanines increases |
| | antigenic |
| 5. HAKAHAHAHAHGHAH- | diversity and |
| | length; reported to |
| | yield high expres- |
| | sion |
| 6. SPHHGG- | Very high affinity |
| | for Cu^{2±} IMAC col- |
| | umn; improved se- |
| | lectivity |
| 7. GluAsp peptide | Non-His domain re- |
| | ported to bind to |
| | IMAC columns, high |
| | selectivity |

### Example 4

To enable quantitative detection of diverse HisTagged proteins, context specificity in the binding of Ab to N-terminal HisTag fusion proteins must be eliminated.

The capability for quantitative detection of diverse proteins using a HisTag FPIA, i.e., the “universality” of the method, is dependent upon the ability of the Ab to bind the HisTag domain with equal affinity regardless of the structure of the protein to which it is fused. If this is not the case, 20 nM of one HisTagged protein might give a decrease in polarization of 60 mP, whereas the same concentration of a different HisTagged protein that binds Ab with lower affinity might only produce only a 30 mP shift. We have hypothesized that interaction of Ab and/or HisTag domains with adjacent protein domains would be the most likely cause of context specificity in the binding of Ab to diverse HisTagged proteins. It follows that taking measures to insure spatial separation between the HisTag domain and the protein to which it is fused will prevent differences in Ab binding like those observed with the three HisTag proteins used in preliminary studies. In order to achieve this, the following experiments were designed:

a) A series of rationally designed linker peptides between the N-terminal HisTag and the proteins to which it is fused are tested to identify the construct that maximizes the accessibility of the HisTag for Ab binding.

b) Different sample preparation methods, including the use of protein denaturants, are examined to develop a general method that minimizes context-specific presentation of the HisTag domain to the Ab.

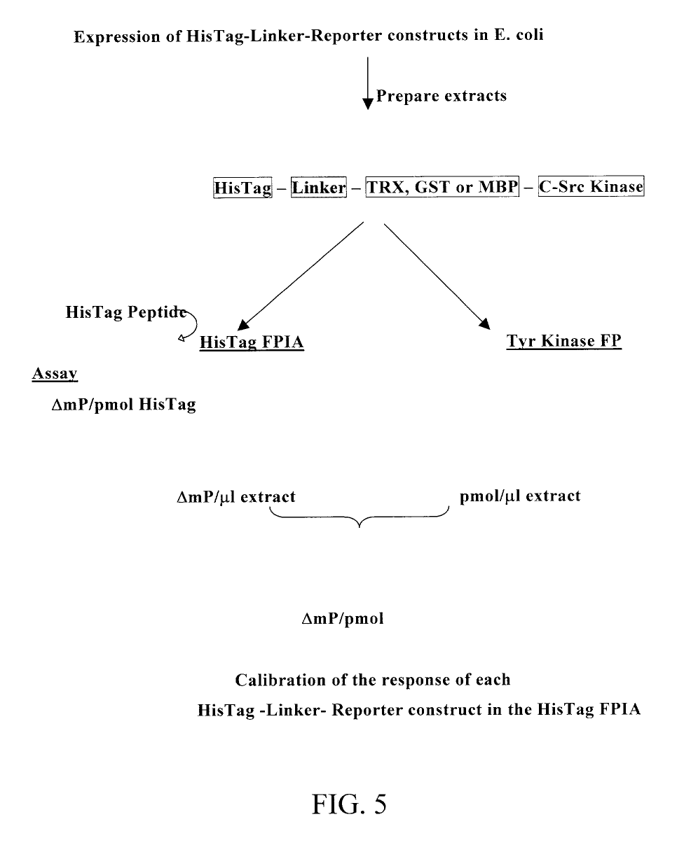

Testing linker peptides to prevent interference with binding of Ab to N-terminal HisTags. The effects of different linker regions on the immunoreactive properties of N-terminal HisTags are tested by expressing a series of HisTag-linker—reporter constructs and comparing their responses in HisTag FPIA with HisTag peptide standards used to calibrate the assays (FIG. 5). The activity of the reporter protein provides an absolute measure of the quantity of the expressed HisTagged proteins; this is necessary because FP is measured in relative units. The reporter domain of these constructs are comprised of one of three different proteins thioredoxin (TRX), glutathione transferase (GST) and maltose binding protein (MBP), fused to the N-terminus of the C-Src kinase (CSK). TRX,GST and MBP were selected as N-terminal domains for the reporter protein because they are commonly used as N-terminal fusions to increase the solubility of eukaryotic proteins in E. coli. In addition, it is important to test the linkers with structurally diverse proteins in order to establish the universality of the assay method: TRX is a small (14 kDa) protein; GST is a homodimer of two 27 kDa subunits, and MBP is a larger (44 kDa) monomer. CSK was chosen as the catalytic domain of the reporter protein for several reasons. It is well expressed in a soluble, active in E. coli (PanVera unpublished) and can be assayed rapidly in crude extracts using PanVera's Core HTS Tyrosine Kinase FP assay (data not shown); the lack of any tyrosine kinases in E. coli extracts eliminates any background noise in the assay. The specific activity of the purified CSK kinase is known, and this is used to calculate the absolute amount of the expressed HisTag-linker-reporter proteins in the crude extracts. This system allows an accurate, quantitative comparison of the FPIA responses of the various HisTag-linker-reporter constructs and the HisTag-peptide standards.

The linker peptides assessed are shown in Table 2 along with a brief description of their fundamental structural properties. Other than preventing interaction with adjacent protein domains, it is difficult to predict how to best present the HisTag as an antigen that would most closely mimic a free peptide, thus a structurally diverse set of linkers are tested. The GS repeats are flexible linkers that are widely used in fusion vectors. The Q-linker and the proline-rich linker regions are naturally occurring sequences of 15-25 amino acids found at the boundaries of functionally distinct domains in a number of E. coli proteins. These domains apparently evolved to provide some spatial separation between two different protein domains, thus are likely to be less tightly associated with folded domains, relatively solvent accessible, and yet resistant to proteolysis. The Q-linker is relatively flexible, whereas the proline rich linker is predicted to be a rigid, extended structure. The α-helical peptide shown in Table 2 is derived from residues 87-97 of troponin C, which is a completely solvent exposed loop on the exterior of the protein. Selection of the best linker peptide is an iterative process, and it is possible that other sequences will work as well or better than those shown in Table 1. One interesting possibility is the last example shown in Table 2, linking of two or more helices with prolines. Prolines introduce a significant bend when present as the first amino acid of a helix, thus the use of different numbers of them should result in the projection of fused HisTags into different orientations relative to the adjacent protein. The construction of the expression vectors and the assay methods used to test the different linkers are described below and above in FIG. 5.

TABLE 2
Representative linker peptide sequences for optimizing antigenic
presentation of N-terminal HisTag fusions.

| Linker peptide | Properties |
|---|---|
| -GS repeats (2-10 aa) | Flexible coil of var. lengths |
| Q-linker (15-25 aa) | Naturally occuring random coil |
| Proline Rich Linker Regions | Naturally occuring, rigid |
| (15-25 aa) | extended |
| KEDAKGKSEEE (11 aa) | Hydrophilic α-helix |
| P-Helix-P-Helix-(20-40 aa) | Helices with bends at junctions |

Complementary oligonucleotides encoding the seven different linker peptides shown in Table 2 are synthesized downstream and in the same reading frame with one or more of the HisTag domains with the most desirable properties as an FPIA tracer; restriction sites are incorporated at both ends to facilitate subcloning. (Depending on the HisTag domain used, some of these oligos are too long to synthesize as a single fragment, and are instead made as two pieces and ligated together.) The annealed double stranded HisTag-linker coding sequences are subcloned as N-terminal translational fusions with each of three different reporter proteins, TRX-CSK, GST-CSK, and MBP-CSK in commercially available pET vectors for high level E. coli expression. The final constructs are translational fusions of the HisTag-linker domains to each of the three different reporter proteins under the control of the strong, IPTG-inducible phage T7 promoter.

The HisTag-Linker-Reporter fusion proteins in pET vectors are expressed in E. coli strain BL21(DE3) using standard methodology for IPTG induction, soluble cell extracts are prepared, and competitive HisTag-FPIA assays are performed using the corresponding Ab/tracer pairs identified in Aims 1 and 2. Briefly, extracts are diluted 5-10 fold into multiwell plates, and Ab and tracer are added in amounts predetermined to be optimal for detection of nanomolar levels HisTag antigens. (A 50 kDa protein expressed at 1 mg/liter will be at a concentration of 20 nM after dilution of the extract into the FPIA assay.) The measured FP values are determined and compared with those from negative control extracts (extracts prepared from BL21(DE3) carrying a pET vector with no insert). The assay is calibrated with unlabeled HisTag-peptide in the presence of control E. coli extracts. The activity of the CSK domains of the reporter fusions is measured in parallel multiwelll assays using PanVera's Tyrosine Kinase FP assay kit. These values are used to calculate the amounts of HisTag-Linker-Reporter present in each extract based on the specific activity of purified CSK kinase. The response of the HisTag-FPIA assay, ΔmP/pmol HisTag antigen, is compared for the fusion proteins and the HisTag peptide calibration standards. These comparisons are used to identify the linkers that present the HisTag antigen in such a way that it binds Ab with the same affinity as the free HisTag peptide calibration standards.

Additional parameters for assay optimization.

Solvent conditions. Some optimization of buffer or additives may be required to eliminate interference in the HisTag-FPIA from endogenous proteins and/or pigments, but we have found that a 3-5 fold dilution of the extracts in the assay is usually sufficient to prevent deleterious effects. However, a more important consideration is how the sample preparation effects the accessibility of the HisTag antigen for Ab binding. If the response of the fusion proteins in the HisTag-FPIA does not correlate with the HisTag-peptide standards, then a number of reagents are tested that can induce conformational changes in proteins, including chaotropic salts such as potassium thiocyanate, detergents such as NP-40 and sodium dodecyl sulfate, organic solvents such as dimethylformamide and dimethylsulfoxide, alcohols including methanol and ethanol.

Suboptimal Ab: We have hypothesized that the context specificity of Ab binding will primarily be a function of the structural relationship between the HisTag and downstream protein domains, however it may also be at least partially dependent on the particular antibody used. Thus several antibodies are generated and tested to identify those with the most suitable characteristics.

The foregoing is considered as illustrative only of the principles of the invention. Further, since numerous modifications and changes will readily occur to those skilled in the art, it is not desired to limit the invention to the exact construction and operation shown and described. Therefore, all suitable modifications and equivalents fall within the scope of the invention.

We claim:

1. A process for detecting proteins in a solution, comprising:
a. attaching a first peptide tag to a protein;
b. attaching a fluorescent molecule to a second peptide tag having the same sequence as the first peptide tag to yield a fluorescent tracer;
c. mixing a binding molecule specific to the tag into the solution with the tagged protein and fluorescent tracer in the solution;
d. detecting the fluorescence polarization of the solution; and,
e. determining the quantity of the tagged protein in the solution.

2. The process of claim 1 wherein the proteins are selected from a crude cell extract.

3. The process of claim 1 wherein the proteins are selected from a subfractionated mixture derived from a cell extract such as a fraction from a chromatographic separation step.

4. The process of claim 1 wherein the proteins are selected from a mixture of proteins resulting from in vitro biochemical synthesis or synthetic chemical methods.

5. The process of claim 1 wherein the tag consists of a polyhistidine.

6. The process of claim 1 wherein the tag consists of a plurality of amino acids which bind to immobilized metal ions.

7. The process of claim 1 wherein the tag is fused to a N-terminus of the protein being expressed.

8. The process of claim 1 wherein the tag is fused to a C-terminus of the protein being expressed.

9. The process of claim 1 wherein the tag is embedded in internal sequence of the protein.

10. The process of claim 1 wherein the tag is attached directly to the protein being expressed.

11. The process of claim 1 wherein the tag is attached with a linker that eliminates context specificity in the interaction of the antibody with the affinity tag.

12. The process of claim 1 wherein the tag does not bind to IMAC adsorbents such as glutathione transferase (GST), maltose binding protein (MBP), thioredoxin (TRX), immunoaffinity domains such as Myc, FLAG, carbohydrate binding domains (MBP, Chitin-binding), and protein binding domains (RNase S-peptide).

13. The process of claim 1 wherein the fluorescent tracer consists of a peptide conjugated to a fluor selected from the group consisting of fluorescein and its derivatives, rhodamine and its derivatives, and fluorescent molecules that can be conjugated to a peptide.

14. The process of claim 1 wherein the binding molecule is bound to the peptide tags with multivalent binding.

15. The process of claim 1 wherein the binding molecule is selected from the group consisting of polyclonal, monoclonal, and recombinant antibodies.

16. The process of claim 1 wherein the binding molecule and tracer are optimized for a quantifiable change in polarization.
